(19) United States
(12) Patent Application Publication
(10) Pub. No.: US 2017/0172227 A1
(43) Pub. Date: Jun. 22, 2017

(54) Temperature-Regulating Garment

(71) Applicant: Cornell University, Ithaca, NY (US)

(72) Inventors: Jintu Fan, Ithaca, NY (US); Huiju Park, Ithaca, NY (US); Yuenshing Wu, Ithaca, NY (US)

(21) Appl. No.: 15/127,073

(22) PCT Filed: Mar. 23, 2015
(86) PCT No.: PCT/US2015/022085

§ 371 Date: Sep. 19, 2016

Related U.S. Application Data
(60) Provisional application No. 61/969,248, filed on Mar. 23, 2014.

(51) Int. Cl. A41D 13/005; A61F 7/02; A41D 13/002; A61F 7/00; A41D 1/00; A41B 9/06

(52) U.S. Cl. CPC A41D 13/0051; A41D 1/002; A41D 13/0053; A41B 9/06; A41D 13/0025; A61F 7/0053; A61F 7/02; A61F 2007/0096; A61F 2007/0234

(57) ABSTRACT

A fabric for temperature regulation and a wearable device for regulating a temperature of a wearer are disclosed. The wearable device has a fabric and one or more microtubes woven in the fabric. Each microtube has an inlet and an outlet, and each microtube is configured to transport a gas through the microtube from the inlet to the outlet. A pump moves gas through the microtube to heat or cool the wearer. The fabric may have a plurality of warp yarns in a warp direction and weft yarns in a weft direction. The fabric also has a plurality of microtubes in a warp and/or weft direction of the fabric. The fabric is formed by interweaving the plurality of microtubes with the warp yarns and weft yarns.

### CROSS-REFERENCE TO RELATED APPLICATIONS

This application claims priority to U.S. Provisional Application No. 61/969,248, filed on Mar. 23, 2014, now pending, the disclosure of which is incorporated herein by reference.

### FIELD OF THE DISCLOSURE

The disclosure relates to wearable heating and cooling devices, specifically wearable garments.

### BACKGROUND OF THE DISCLOSURE

It has been reported that space heating and cooling of buildings represents more than 13% of all energy used in the United States. The electricity usage of commercial and residential buildings accounts for more than 70% of all electricity used in US. This represents 40% of US's total energy bill, and contributes to almost 40% of the US's carbon dioxide emissions. The large energy consumption associated with space heating and cooling is primarily driven by the need to provide a comfortable range of temperatures to the building's occupants. In practice, the neutral band is usually between 71° and 75° F., the temperature set points between which no action is taken by the building's heating and cooling systems. If this neutral band can be expanded by as little as 4° F. (or ˜2° C.) in each direction, over 15% of energy saving is possible, accounting for over 1% of total's energy use.

There have been substantial developments in smart thermal regulatory clothing through material innovation, creative clothing design and incorporation of actuators or active cooling/heating elements. For example, some temperature sensitive membranes rely on the chemical properties of a membrane. One category of temperature sensitive membranes is fabricated with soft and hard polymer segments. The temperature-sensitive property is a result of the crossing of a transition temperature in the material such as the glass transition temperature or the melting point of crystalline segments. It was claimed that these materials increase water vapor transmission rate with increasing temperature. However, the observed increase in the water vapor flux across the transition temperature may be due to the increased vapor pressure gradient rather than the increase in vapor permeability.

Another type of temperature sensitive membranes is made by grafting a temperature sensitive polymer [e.g., Poly-N-isopropylacrylamide (PNIPAAm)] onto a microporous membrane. Below the low critical soluble temperature (LCST), the swelling the temperature sensitive polymer block the pores of the membrane (off status), and above the LCST temperature sensitive polymer shrink and open the pores of the membrane (on status). However, to make such a temperature sensitive membrane work, the material should be in a wet state and the temperature change should be sufficiently large, which is generally not the case in indoor environment.

Another category of smart thermal regulatory clothing is variable fabric construction. For example, some developed fabric material opens its structure when it is wet, allowing greater permeability to moisture transmission under sweating condition. A moisture responsive fabric was also developed by incorporating both moisture responsive and non-responsive yarns in one fabric and the expansion of moisture responsive yarns create openings in the fabric. These materials are advantageous only when the wearer is in profuse sweating condition, which is generally not the case in the indoor environment.

Another category of smart thermal regulatory clothing is moisture management fabrics. Moisture management fabrics can be produced by using moisture wicking fibers and multilayer fabric structure. Engineered fibers with non-circular cross-section such as Coolmax facilitate the wicking of sweat away from the skin through capillary action. Multilayer fabric structures with a next-to-skin layer made of hydrophobic fibers and outer layer made of hydrophilic fibers can help the wicking of sweat away from the skin. Recently, a novel fabric structure was developed that was inspired from the branching structure of trees, in which yarns are grouped together in side facing the skin and split into individuals in the side away from the skin. The material has a directional liquid transport property. However, moisture management fabrics do little to regulate temperature in the indoor environment.

Another category of smart thermal regulatory clothing is phase change materials (PCM). PCMs have been incorporated into the textile fabrics by embedding PCM microcapsules into the fibers, coating PCM microcapsules onto the fabric surface, or filling PCM into the hollow fibers. PCM containing textile fabrics absorb when the temperature rises above the phase transition temperature of PCM and release heat when the temperature decreases below the phase transition temperature. PCM is only effective within a short period during which the environmental temperature changes. It is not applicable for long exposure in the indoor environment.

Another category of smart thermal regulatory clothing is garment incorporating shape memory alloy (SMA). NiTi two-way SMA helical coils were incorporated into a cold weather jacket for changing the thermal insulation in response to the environmental conditions. The flat coils at 30° C. were expanded at both 0 and −5° C. spontaneously, creating an additional air layer between the adjacent layers of the clothing system. Consequently, the clothing provides an improved buffering effect for the increase in thermal insulation upon a sudden drop of environmental temperature. Wearers with such clothing reported a warmer feeling, but not statistically significant except at the moment of the transition point. This may be caused by the increased heat conduction through the metal alloy. Furthermore, the alloy also increases the undesirable weight of the clothing.

Another category of smart thermal regulatory clothing is garment designed to promote ventilation. Ventilation promotes exchange the hot & moist air within the microclimate next to the skin and cold & dry air in the environment. Clothing has been specifically designed to facilitate ventilation by creating space between the clothing and the skin as well as having ventilation openings at appropriate locations. A “chimney” cooling effect is created as the garment moves because of body motion or external wind. Such garments can help the wearer stay cooler in the hot environment or when the wearer is playing active sports, however they may not be welcomed in the office environment for their peculiar appearance. Furthermore, body motion can create more heat than the additional cooling effect gained.

Another category of smart thermal regulatory clothing is clothing incorporating a cooling fan. Some jackets incorporate built-in light electric fans. The fans at the back pump fresh air around the wearer and out through the neck and sleeve ends to help cool the wearer. The jacket is very effective in cooling, but has an obvious drawback of the balloon effect caused by the airflow.

Another category of smart thermal regulatory clothing is clothing with heating elements. Heating is often much easier than cooling. Heating elements have been incorporated into clothing using conductive yarns made of different materials (e.g. conductive polymers, carbon fibers, metallic coated fibers, electric wires, etc.). However, heating elements alone cannot regulate temperature, for example, in warm environments.

Previous designs fail to produce a wearable device that can be comfortably integrated into a garment and also regulate the temperature of a wearer in an indoor environment. For example, U.S. Pat. App. Pub. No. 2015/0033437 describes a temperature adjustable air-cooled undergarment using a network of macrotubing to distribute cool air to the wearer. However, the macrotubing is too large to be woven into the fabric in a way that would be flexible and comfortable to the user. Furthermore, the length of tubing in this design creates an undesirable pressure drop in the macrotubes. Because there is no need to create a greater pressure drop in the macrotubes, this design uses a fan or compressor to move air through the macrotubes. In this design, the wearer may be tethered to the compressor which is inconvenient if the wearer needs to move from one workspace to another

In another example, U.S. Pat. App. Pub. No. 2012/0260398 describes a personal cooling apparatus. Like the previously discussed design, this design also uses macrotubes to distribute temperature-controlled air. Again, the macrotubing is too large to be woven into the fabric in a way that would be flexible and comfortable to the user. In addition, the macrotubes simply terminate midway through the garment. Furthermore, this design requires multiple fabric layers (an inner layer and an outer layer with the macrotubes in between). As such, the garment is rigid, bulky, and uncomfortable for the wearer. When air moves through the macrotubing, this design would create an undesirable balloon effect in between the inner and outer layers.

Therefore, there remains a need for a flexible, lightweight, and comfortable temperature-regulating garment and thermoregulatory clothing system to enable expansion of the neutral band for buildings without compromising the comfort, wearability, washability, appearance, and safety of the wearer's clothing.

### BRIEF SUMMARY OF THE DISCLOSURE

Some embodiments of the present disclosure may be described as a wearable device for regulating a temperature of a wearer. The device may comprise a fabric. The fabric may be configured to be worn by the wearer, for example, as a piece of clothing or undergarment. In some embodiments, resistive wires may be positioned in the fabric, and the one or more resistive wires may be configured to heat the wearer.

The device may further comprise one or more microtubes woven into the fabric. Each microtube may have one or more inlets and outlets. Each microtube may be configured to transport a gas through the microtube from the inlet to the outlet. In some embodiments, one or more microtube outlets may be exposed to ambient air. In some embodiments, the one or more microtube outlets may be bundled together. The one or more microtubes may be formed from an elastic material and/or a material conductive to heat, such as a heat conductive polymer. The microtubes may be less than 2 mm in diameter. The microtubes may be porous, and the microtube outlets may be a plurality of pores.

The device may further comprise a pump. The pump may have an intake and an outlet. In some embodiments, the intake of the pump may be exposed to ambient air. The pump may be configured to move a gas. In some embodiments, one or more microtube inlets may be in fluidic communication with the outlet of the pump.

The device may further comprise a microcontroller in electronic communication with the pump. In some embodiments the pump is controllable by the microcontroller. The device may further comprise a temperature sensor (such as a thermocouple) in electronic communication with the microcontroller. The temperature sensor may be configured to be positioned near the skin of the wearer. In some embodiments, the microcontroller is configured to regulate the pump according to a signal received from the temperature sensor. For example, the microcontroller may regulate the pump by regulating a speed of the pump. The microcontroller may also be configured to communicate with an environmental system. The device may further comprise a battery or other power source configured to provide energy to the pump, microcontroller, and/or the temperature sensor. The battery/power source may be rechargeable through the wearer's motion.

In some embodiments, the device may further comprise an energy conversion device. The energy conversion device may be in fluidic communication with one or more microtube outlet as well as the intake of the pump.

Some embodiments of the present disclosure may be described as a fabric for temperature regulation. The fabric may comprise a plurality of warp yarns in a warp direction. The fabric may further comprise a plurality of weft yarns in a weft direction. One or more of the warp yarns or weft yarns may be conductive to heat. The fabric may further comprise a plurality of microtubes in a warp and/or weft direction of the fabric. Each microtube may have one or more inlets and outlets. Each microtube may be configured to transport a gas through the microtube from the inlet to the outlet. The fabric may be formed by interweaving the plurality of microtubes with the warp yarns and weft yarns as the fabric is being made. The microtubes may be formed from an elastic material and/or a heat conductive polymer. The microtubes may be less than 2 mm in diameter. The microtubes may be porous, and one or more of the microtube outlets may be a plurality of pores.

One embodiment of the present disclosure is configured as an unobtrusive undershirt with thermal regulatory function (i.e., cooling or heating depending on the physiological condition of the wearer). The undershirt is embedded with temperature sensors to monitor the temperature next to the skin. If the next-to-skin temperature is too high, the cooling function of the undershirt is activated, and conversely if the next-to-skin temperature is too low, the heating function is activated. The cooling function is achieved by circulating cold air through the microtubes embedded, for example, by knitting or weaving, in the undershirt. The cold air may be pumped directly from the environment if the ambient air is colder and drier. In another embodiment, the air may be cooled using an energy conversion device (for example, flexible thermoelectric device and flexible heat pipe) before being pumped into the microtubes in the undershirt. The heating function may be achieved through circulating warm air through the microtubes woven in the undershirt and/or by electrical heating using conductive yarns embedded in the undershirt. The power for the thermal regulatory function of the undershirt may be supplied through a portable battery, which can be charged by, for example, wireless inductive charging.

### DESCRIPTION OF THE DRAWINGS

For a fuller understanding of the nature and objects of the disclosure, reference should be made to the following detailed description taken in conjunction with the accompanying drawings, in which:

FIG. 1 is an illustration showing a front view of one embodiment of the present disclosure as worn by a mannequin;

FIG. 2 is an illustration showing a back view of the embodiment of FIG. 1;

FIG. 3 is an illustration showing a back view of the embodiment of FIG. 1 beneath a dress shirt;

FIG. 4 is an illustration showing a front view of the embodiment of FIG. 1 beneath a dress shirt and jacket;

FIG. 5 is an illustration showing a back view of the embodiment of FIG. 1 beneath a dress shirt and jacket;

FIG. 6 is an illustration showing a front view of the embodiment of FIG. 1 beneath a dress shirt and tie;

FIG. 7 is a table of experimental results using the embodiment of FIG. 1;

FIGS. 8-11 are illustrations showing numerical simulations to predict the additional heating or cooling power required to maintain thermal comfort;

FIG. 12 is an illustration showing air flow in a front view of one embodiment of the present disclosure;

FIG. 13 is an illustration showing pressure differentials in different areas of a temperature-regulating garment according to one embodiment of the present invention; and

FIG. 14 is an illustration of an energy conversion unit having flexible thermoelectrics.

### DETAILED DESCRIPTION OF THE DISCLOSURE

The present disclosure can be embodied as a wearable device such as a garment or an undergarment. The wearable device comprises a fabric configured to be worn by a wearer. The fabric may be conductive to heat. The fabric may comprise natural or artificial fibers, or a combination thereof The fabric may have multiple layers.

The device further comprises one or more microtubes positioned in the fabric, each microtube having an inlet and an outlet. The microtubes are configured to transmit a gas through the microtube from the inlet to the outlet. In some embodiments, the microtubes may be porous, and the pores of the microtubes are one or more outlets. The microtubes may be less than 2.0 mm in diameter. In some embodiments, the microtubes may be less than 1.0 mm, 0.5 mm, 0.1 mm, 0.05 mm, or 0.01 mm in diameter. Microtubes of other diameters may be used provided that the microtubes are small enough to be woven. The microtubes may be woven into the fabric or embedded into the fabric as inlays during knitting. In some embodiments, the microtubes may replace one or more warp and/or weft yarns of the weave of the fabric. In some embodiments, the microtubes may be woven, or threaded, through the weave of the fabric. The microtubes may also be inserted into a knitted structure as inlays. The microtubes may also be positioned between layers of the fabric. The microtubes may be fixed in the fabric in a variety of patterns. For example, microtube density (an amount of microtube coverage of an area of fabric) may be higher in certain areas of the undergarment, such as the underarms, small of the back, neck, etc.

In one embodiment, the microtubes are inserted into the knitted structure through laying-in or weft insertion techniques in either weft or warp knitting. For example, the weft yarn (i. e., inlaid yarn) may be fed through a tube inlay feeder attached to a feeder guide. The inlay yarn may be fed in advance of cylinder and dial needles moving out to clear for the knitting yarn. After the cylinder needle is raised up, the inlay yarn is then trapped in-between the cylinder and dial face loops. By manipulating the movement of the needles and feeding of inlay yarn, the inlay yarn can also be interlaced with the knitting structure. In warp knitting, when the needles are in the lowered position during a knitting cycle, a so-called ‘open-shed’ effect is created at the back of the machine. A weft yarn can then be laid across the full width, to be carried forward by special weft insertion bits over the needle heads and deposited on top of the overlaps on the needles and against the yarn passing down to them from the guide bars. in this way, the inserted weft becomes trapped between the overlaps and underlaps. Multiple weft yarns may be supplied simultaneously from a stationary creel to an insertion carriage. The multiple weft yarns may be simultaneously laid onto a conveyer to be fed individually to the knitting machine. There can be many variations of laying-in and weft insertion in warp knitting. An inlaid yarn may pass across part or all of the knitting width or it may be introduced in different directions.

Existing laying-in and weft insertion techniques may need to be modified to accommodate the insertion of microtubes since the microtubes tend to be thicker than the inlay yarns commonly used in the industry. Processing parameters, such as needle size, needle gauge, knitting structure (combinations of knit, tuck, and float loops), loop length, size of the knitting (yarn count), and tension of the knitting yarns may need to be modified for microtubes of the present disclosure.

The microtubes are configured to carry a gas, such as air. Other gases may be used. The microtubes may advantageously allow heat to transfer between the gas flowing through the microtubes and the wearer. For example, if the gas flowing through the microtubes is warmer than the wearer is, the microtubes should allow heat from the gas to be transferred to the wearer. Conversely, if the wearer is warmer than the gas flowing through the microtubes, the microtubes should allow heat transfer from the wearer to the gas. The microtubes may be formed from an elastic material. The microtubes may be formed from a polymer, such as a heat-conductive polymer.

In one embodiment, a single microtube may be positioned in the fabric. In another embodiment, multiple microtubes may be positioned in the fabric. In embodiments with multiple microtubes, the lengths of two or more of the microtubes may be the same or different. The diameter of two or more of the microtubes may be the same or different. For example, a larger diameter microtube may be positioned in the fabric in a location known to be warmer or colder than the average temperature. The microtubes may each have different cross-sectional shapes—e.g., oval, circular, etc. The cross-sectional shape of a microtube may vary along its length.

In one embodiment, the contact between the microtubes and the skin may be maximized. For example, the fabric may be knitted in such a way that compresses the fabric against the skin for increased contact. The fabric may be knitted with blend yarns containing hygroscopic fibers (e.g., cotton) and stretchable fibers (e.g., Lycra). White the hygroscopic fibers absorb moisture to keep the skin dry, the stretchable fibers provide mild compression to the skin, creating a form-fitting shape to the body. To reduce the air circulation resistance, multiple microtubes connected in parallel may be used. In one embodiment the garment may be made of multiple panels (e.g., front and back panels) to improve fitting. The multiple panels can be joined by flatlock stitches which provide high strength and soft touch on the skin, and prevent the microtubes from being puncture or damaged by the sewing needle during construction due to their very narrow seam allowance (for example, ⅛ inch).

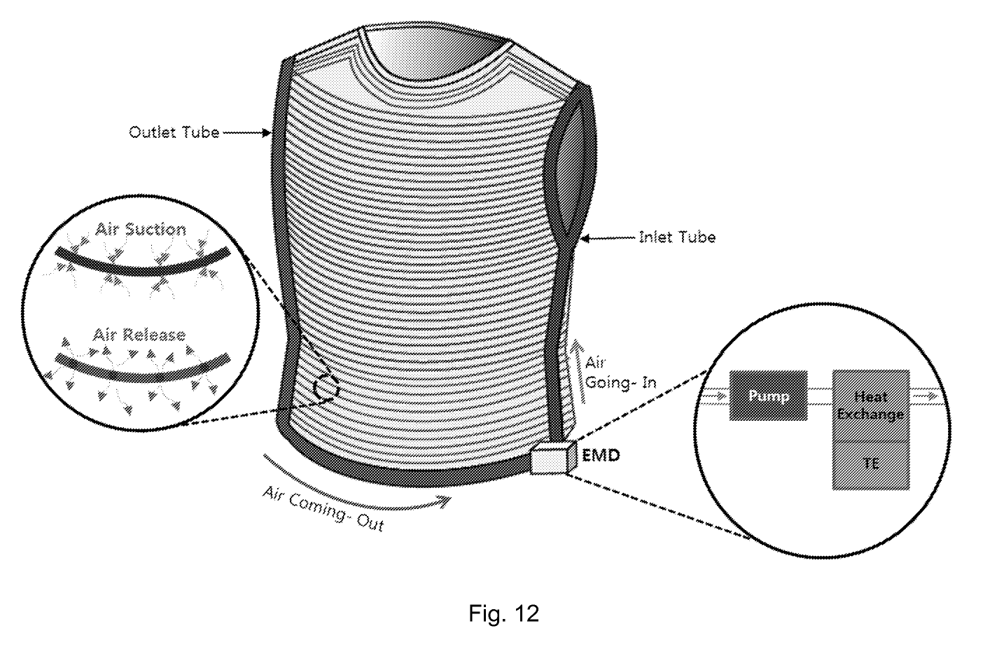

In some embodiments, the microtubes are porous. In these embodiments, a portion of the gas travelling through the microtubes escapes the microtubes through the pores in order to cool or heat the wearer. The pores may be configured such that gas escapes throughout the length of each microtube. For example, see FIG. 12.

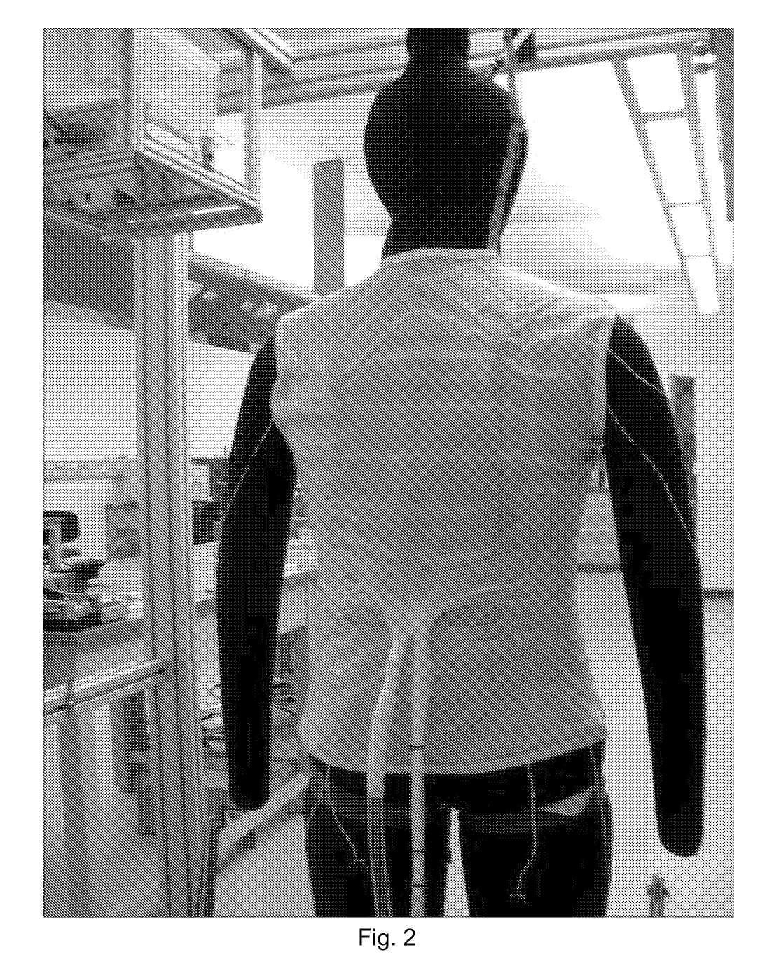

The inlets of the microtubes may be bundled together (see, e.g., FIG. 2). In this way, the microtubes may be positioned in the fabric (such that the inlets of each microtube are located in proximity to one another, for example such that the microtubes are substantially parallel with one another for a length near the inlet. The inlets of each microtube may be bundled such that a fluidic connection can be established with an outlet of a pump or other component (e.g., localized gas source, heater, cooler, etc.)

The device may further comprise a pump having an intake and an outlet. The pump is configured to move a gas. The intake of the pump may be exposed to ambient air. More than one pump may be used. Each microtube inlet may be in fluidic communication with the outlet of the pump. In some embodiments, the outlet of each microtube is in fluidic communication with the intake of the pump. The microtubes and pump may be configured as a closed circuit where the inlet and the outlet of each microtube are in fluidic communication with the outlet and intake of a pump, respectively. Gas is recirculated through a closed-circuit device. In closed-circuit embodiments, the gas may be heated or cooled, as appropriate, while being recirculated.

In other embodiments, each microtube outlet may be exposed to ambient air. As such, the microtubes and pump create an open circuit. The pump receives ambient air and moves the ambient air through the microtubes in an open-circuit device. In an open-circuit device, the ambient air may be cooled or heated before entering the microtubes. In some embodiments, humidity may be added or removed from the ambient air before entering the microtubes.

In one embodiment, the one or more microtube outlets are bundled together. For example, the microtubes may be positioned in the fabric such that the outlets of each microtube are located in proximity to one another. The microtube outlets may be located outside of the fabric—i.e., on a side of the fabric opposite the wearer. In this way, the outlets of the microtubes may be physically bundled and the bundle of microtube outlets can be placed in fluidic communication with an intake of pump. The bundled outlets may simply be exposed to ambient air without attachment to a pump or other component. In other embodiments, the outlets of each microtube may be unbundled. In this way, the outlets of the various microtubes may be placed at different locations of the garment. In some embodiments, some microtube outlets may be bundled together, while others are not.

The device may further comprise a temperature sensor configured to provide a temperature signal. The temperature sensor may be positioned near the skin of the wearer. The temperature sensor may be a thermocouple or other temperature sensor known in the art. More than one temperature sensor may be used. Temperature sensors may be placed in various locations of the garment. For example, in an undershirt according to an embodiment of the present disclosure, temperature sensors may be placed at locations corresponding to the wearer's front, back, side, and underarms.

The device may further comprise a microcontroller in electronic communication with the temperature sensor and/or the pump. The microcontroller is configured to regulate the temperature of the wearer by, for example, altering the speed of the pump based on the temperature data of the temperature sensor. The microcontroller may interface with an environmental system as is further described below under the heading of “Further Exemplary Embodiments.”

The device may further comprise a heater to heat the gas of the microtubes and/or the device may further comprise a cooler to cool the gas of the microtubes. The device may further comprise an energy conversion device to recover energy that would otherwise be wasted. In some embodiments, the energy conversion device is in fluidic communication with each microtube outlet and the intake of the pump. The energy conversion device may be, for example, a heat exchanger. In other embodiments, the energy conversion device may be a flexible thermoelectric device, a flexible heat pipe, or other such devices known in the art.

The device may further comprise a battery to provide energy to the pump, microcontroller, and/or the temperature sensor. The battery may be rechargeable. In some embodiments, a charger may be configured to recharge the battery. For example, the battery may be recharged by way of a charger that converts the wearer's motion into electrical energy.

The device may further comprise one or more resistive wires positioned in the fabric. The one or more resistive wires may be configured to heat the wearer. The heat provided by the resistive wires may be controlled by the microcontroller.

### Further Exemplary Embodiments

One embodiment of this disclosure is directed to an unobtrusive undershirt with thermal regulatory function (i.e., cooling or heating depending on the physiological condition of the wearer). The undershirt includes temperature sensors to monitor the temperature next to the skin. If the next-to-skin temperature is too high, the cooling function of the undershirt is activated, and conversely if the next-to-skin temperature is too low, the heating function is activated. Embodiments of the undershirt may provide heating, cooling, or both heating and cooling.

The cooling function is achieved by circulating cold air through microtubes woven in the undershirt. The cold air may be pumped directly from the environment if the ambient air is colder, or the air may be cooled using an energy conversion device (for example, flexible thermoelectric device and flexible heat pipe) before being pumped into the microtubes in the undershirt. The heating function is achieved through circulating warm air through the microtubes woven in the undershirt and/or electrical heating of conductive yarns (i.e., resistive wires) woven in the undershirt. The power for the thermal regulatory function of the undershirt may be supplied through a portable battery, which can be charged through, for example, wireless inductive charging.

The microcontroller of the air-conditioning undershirt may be interfaced with the indoor air-conditioning system to regulate the set temperature and humidity of the indoor environment. For example, the microcontroller may communicate with a centralized system that controls the temperature and humidity of an entire building, or certain sections of a building. For example, the centralized system may communicate to the microcontroller (or vice versa) if the wearer enters a new environmental zone. In one embodiment, the microcontroller can alert the centralized system that a wearer has entered a previously unoccupied environmental zone and change the parameters of the environmental zone or the wearer's device accordingly. In another embodiment, the centralized system may increase the temperature of circulated gas when the wearer steps into a freezer. In one embodiment, the temperature regulation system of the undershirt may be interfaced with mobile phones and indoor temperature control devices.

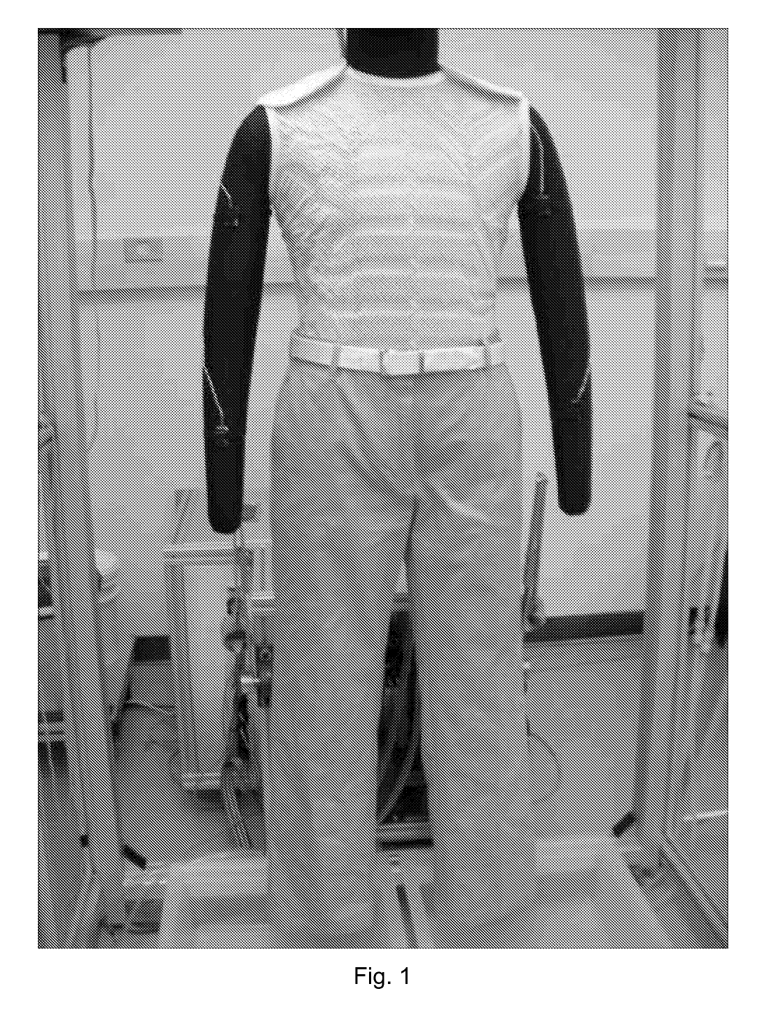

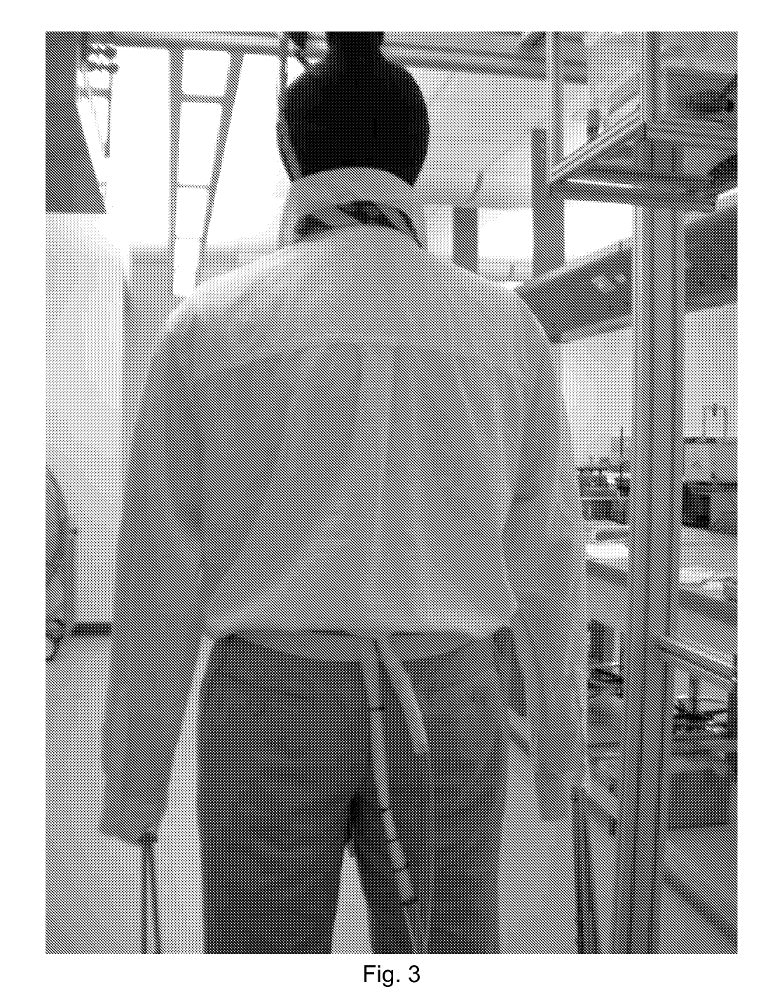

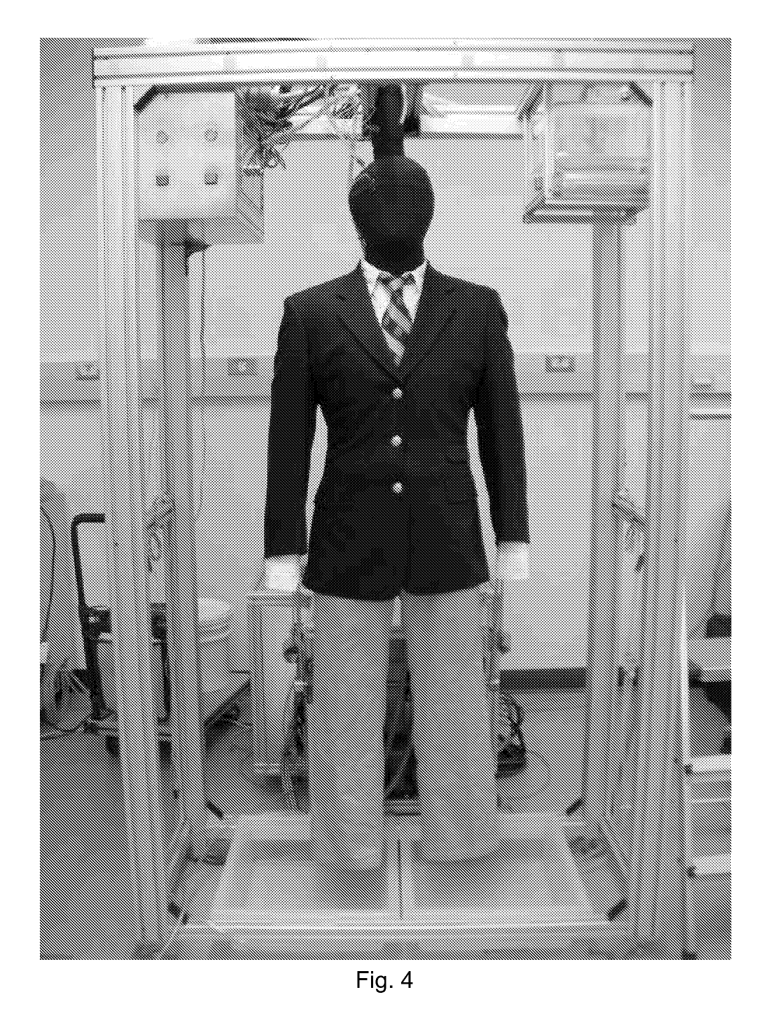

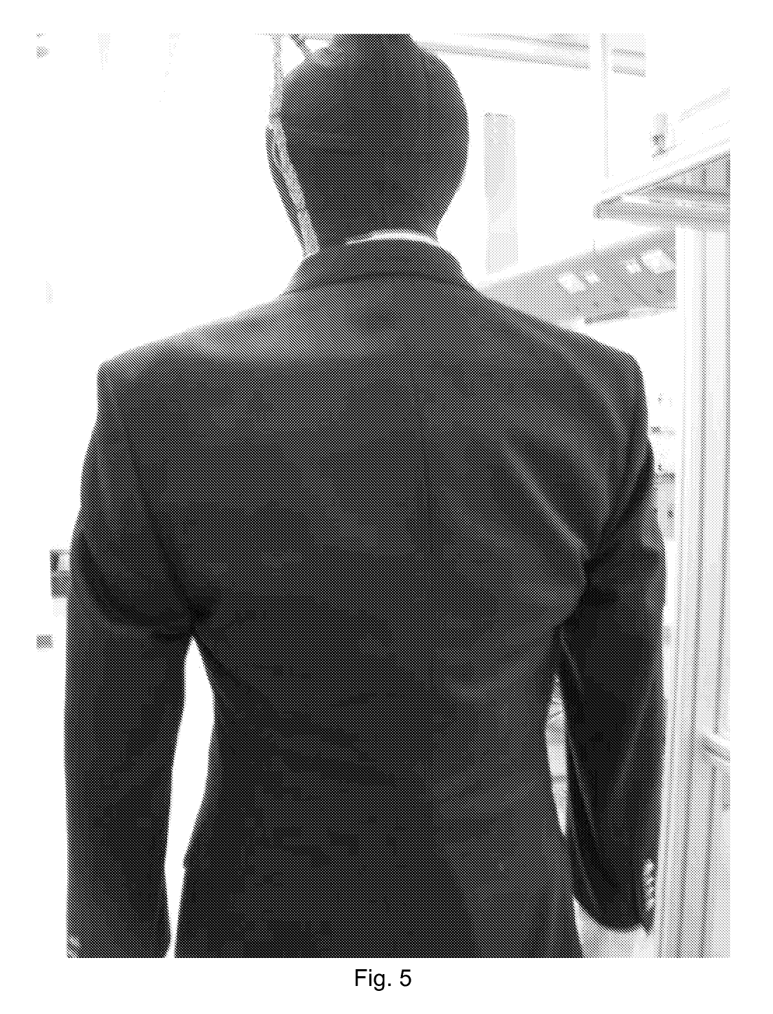

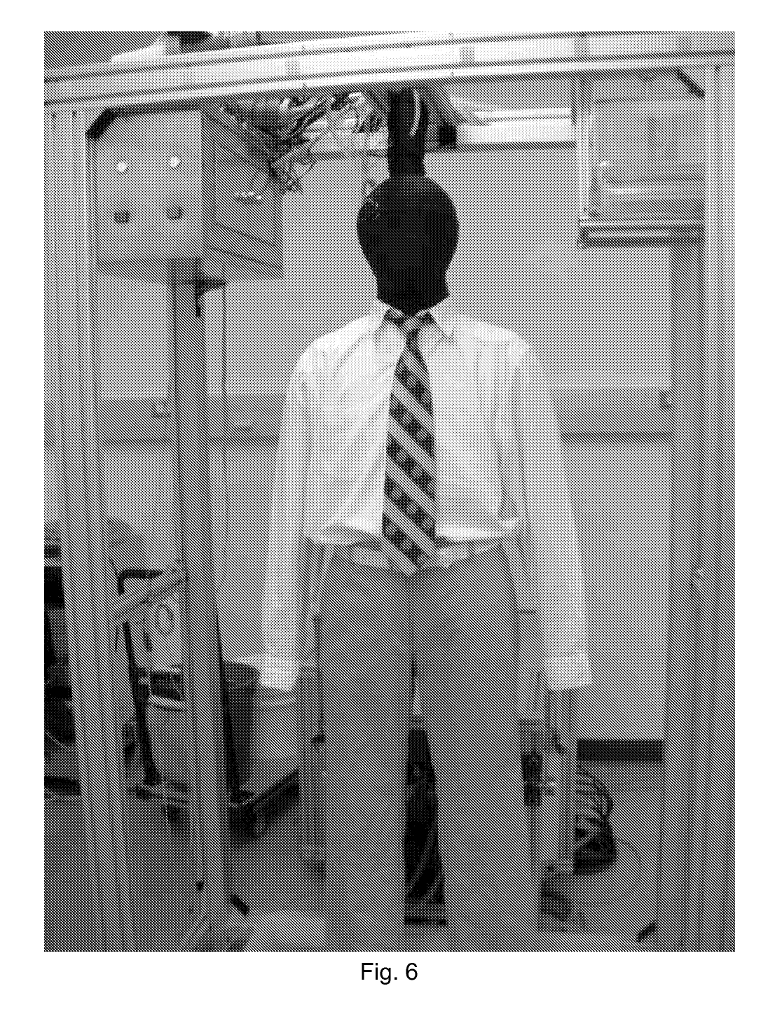

FIGS. 1-6 show a prototype air-conditioning undershirt and how it is dressed under a dress shirt and jacket. The entire system of an exemplary air-conditioning undershirt comprises of the following components:

Flexible microtubes embedded into the undershirt fabric, portable micro-pumps or localized compressed air source for generating air circulation, energy conversion device (for example, flexible thermoelectric device and flexible heat pipe) for cooling and/or heating the air being circulated in the microtubes, temperature sensors embedded in the undershirt to monitor next-to-skin temperature, and a microcontroller interfaced with the temperature sensors, micro-pumps, energy conversion devices and indoor air-condition system to regulate the cooling and heating power of the energy conversion device, speed of air circulation (viz. pumping rate of the pumps) and the set points (viz. temperature and humidity) of indoor air-condition system (if used in indoor environment) depending on the next-to-skin temperature. Controlled heating can be done by heated warm air circulating in the microtubes in the undershirt. The undershirt may also comprise a portable battery, which can be charged through wireless inductive charging and connection to a local power sources if needed.

In order to improve the cooling/heating efficiency of the device, one may enhance the heat exchange between the body of the wearer and the undershirt by, for example, configuring the microtubes to cover as large body surface area as possible. Another improvement uses elastic yarns in the base fabric and elastic polymers in the microtubes to improve fitting of the undershirt to the body. Another improvement involves configuring the design of the undershirt to have better contact with the contour of the body. Another improvement involves using conductive yarns (e.g. conductive carbon fibers yarns, conductive polymer yarns, metallic yarns, etc.) in the base fabric of the undershirt to improve heat transfer.

It may be advantageous to improve the heat exchange between the microclimate next to the skin and the circulating air through the microtubes, for example, by using the heat conductive polymer to make the microtubes. In another embodiment, the cross-section of the microtubes may be configured to increase the surface area for heat exchange. For example, the cross-section of the microtubes may be non-circular. In another example, only certain portions of the microtubes may have a non-circular cross-section. In one example, the diameter of a circular cross-section in one portion of the microtube may be different from other portions of the microtube. In some embodiments, the microtubes may be perforated so that cool or warm gas may be released into the microclimate next to the skin to enhance heat exchange.

The general wearing comfort should also be considered in constructing the device, which includes tactile comfort, breathability, mobility, and weight. Soft and hygroscopic fibers like cotton and silk can be used for making the base fabric for better tactile comfort and moisture absorption. Moisture management and breathable fabric structure can be used for keeping the skin dry. The undershirt may also be designed in such a way so that it can be put on and off easily.

It may be advantageous that the entire air-conditioned undershirt is lightweight and unobtrusive when worn underneath the outer garments. All components including the base fabric, microtubes, micro-pump, microcontroller, temperature sensors, energy conversion device, portable battery and connections can be configured to be as light and thin/small.

The aftercare of the undershirt is also an important consideration. For washability, all mechatronics (viz. the micro-pump, sensors, energy conversion device and microcontroller) should be detachable before washing.

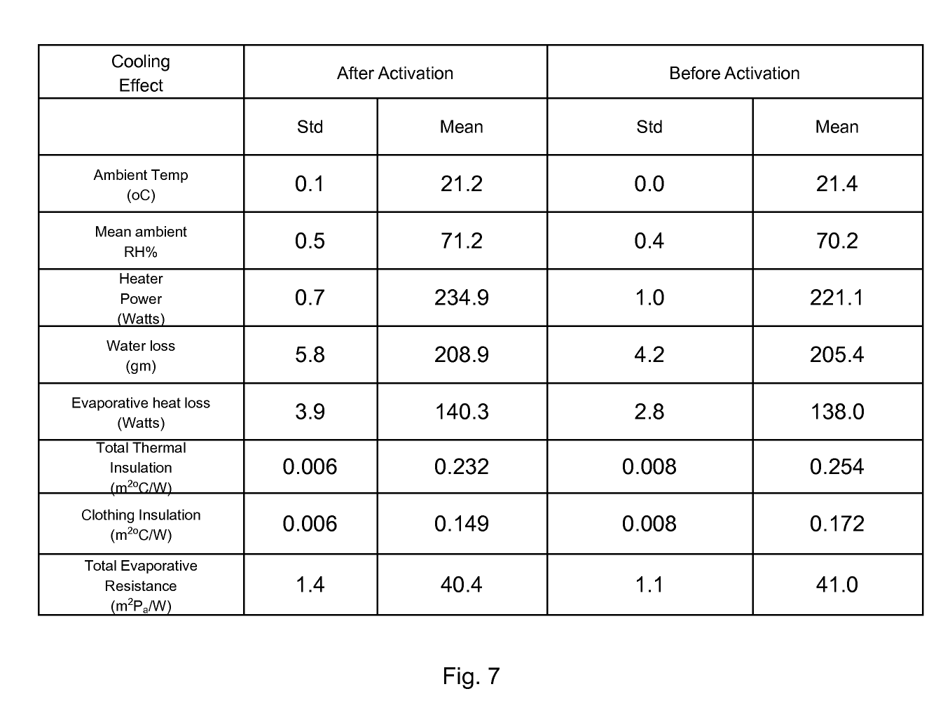

A prototype air-conditioning undershirt has been constructed and tested on a sweating fabric manikin-Walter. The undershirt was worn underneath a dress shirt and formal jacket, simulating a wearer in an office environment. Ambient air from a 20° C. and 65% relative humidity environment was pumped through the microtubes woven in the undershirt. The results are listed in FIG. 7. It was found that circulating ambient air around the microtubes create 13.8 Watts cooling to the body or have an equivalent 13.4% reduction in the intrinsic thermal insulation of entire clothing system. This experiment demonstrated the potential of this device in creating 24 Watts (15 Watts/m²) of additional cooling or the equivalent 20% reduction in the intrinsic thermal insulation required to increase the set temperature of the indoor environment by 2° C. while still maintaining thermal comfort.

The disclosure may be implemented as, but not limited to, the following versions:

Instead of heating through circulating warm airs in the microtubes woven in the undershirt, heating may be carried out by supplying electrical power to the conductive yarns in the undershirt.

Instead of charging the portable battery using wireless inductive charging, power can be harvested from exposure to sunlight through photovoltaics, from body motion through piezoelectric materials, etc.

The portable battery may be a flexible battery embedded in the fabric.

The micro-controller and other auxiliary devices of the undershirt may be interfaced with mobile phones, furniture and/or other surroundings for easy user-interface, additional control and supply of air and power.

In some embodiments, the microtubes are in fluid communication with a localized gas source. The localized gas source may provide, for example, a warm gas or a cool gas. The localized gas source may be pressurized. In some embodiments, a gas is provided from a pressurized gas source and the gas is cooled upon its expansion when released from the pressurized source.

The gas used in embodiments of the present disclosure may be air or any other gas as will be apparent in view of the disclosure.

The device can be used in extreme environments (i.e., very hot or very cold) environment to protect the wearer (viz. assisting the wearer to maintain thermal comfort) and improve his/her performance The device can therefore be used for outdoor and/or performance clothing (ski wear, firefighter uniform, sportswear, activity wear) and military uniforms.

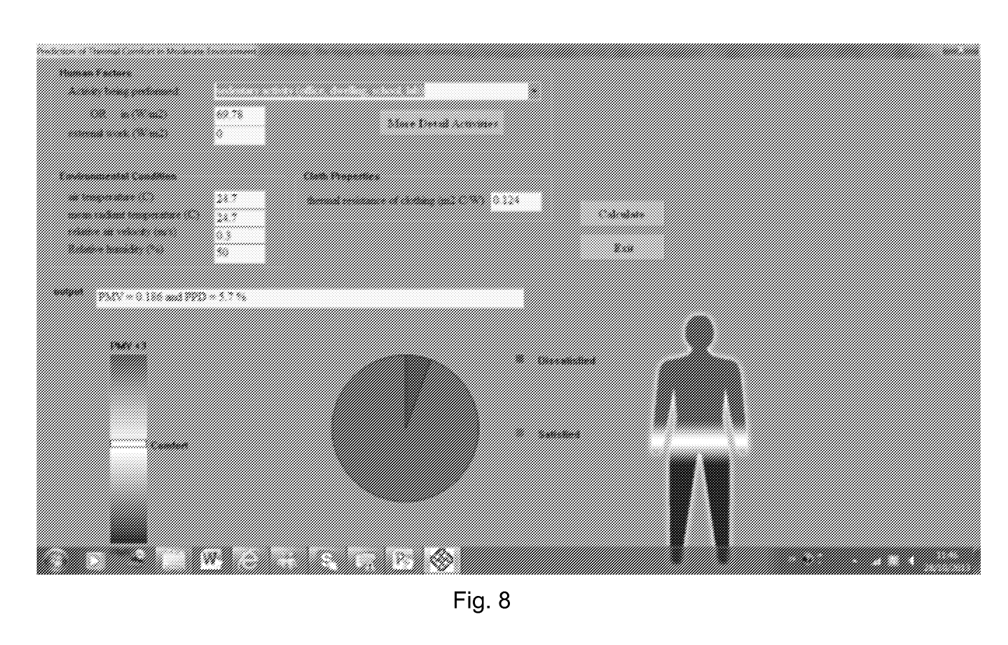

Based on the physiological model established in ISO 7730, numerical simulations were conducted to predict the additional heating or cooling power required to maintain thermal comfort of an average person when the neutral band is expanded in either direction. The results are shown in FIGS. 8-11. The simulation shows that the expansion of 2° C. in the neutral band in each direction is equivalent to additional 15 Watts/m² heating or cooling from clothing or 20% change of clothing thermal insulation.

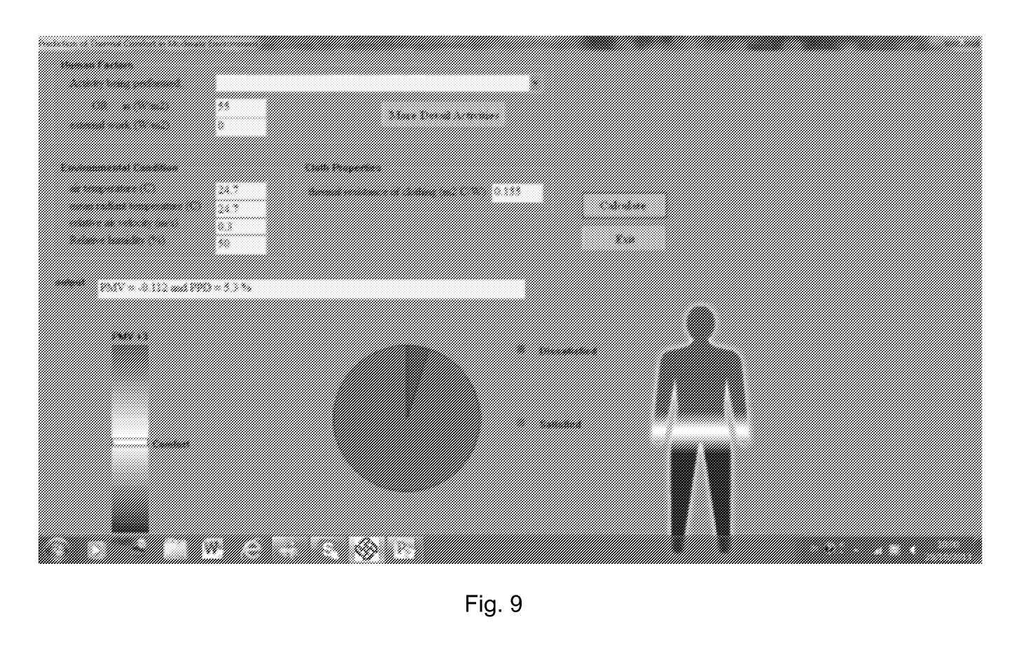

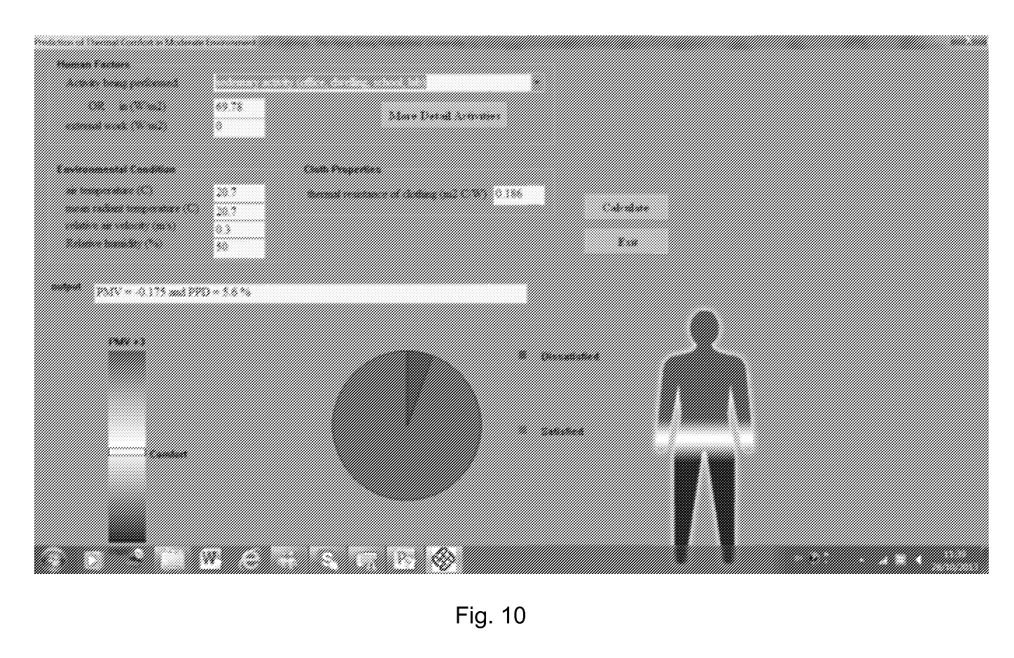

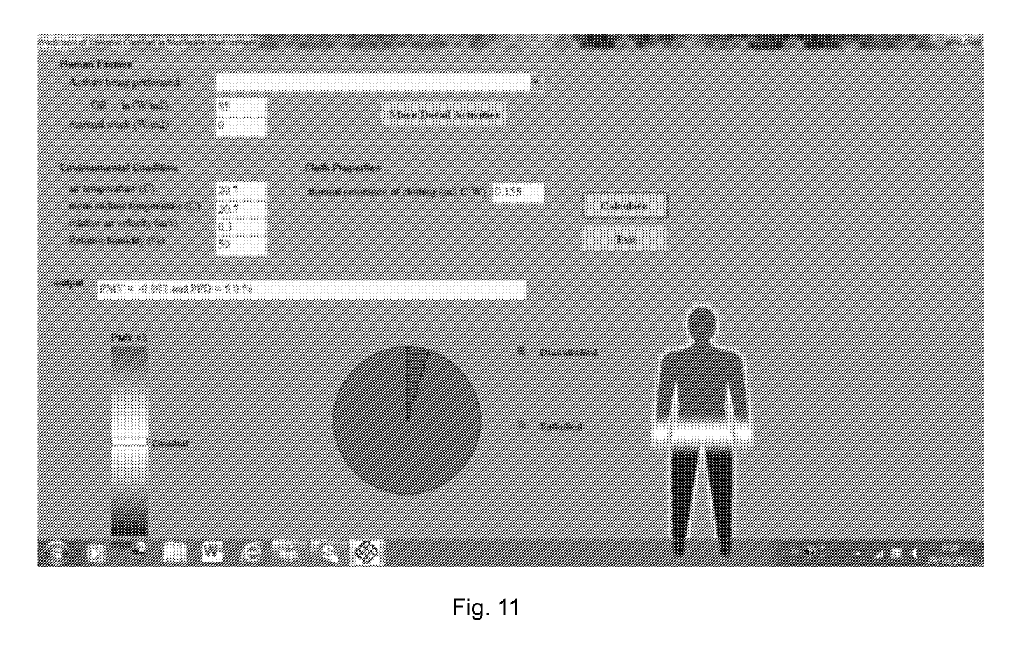

FIG. 8 shows a 20% decrease in thermal insulation corresponds to 2° C. increase in optimum set point. FIG. 9 shows a 15 W/m² (24 Watts) cooling corresponds to 2° C. increase in optimum set point. FIG. 10 shows a 20% Increase in thermal insulation corresponds to 2° C. reduction in optimum set point. FIG. 11 shows a 15 W/m² (24 Watts) heating corresponds to 2° C. increase in optimum set point.

FIG. 12 shows air flow in one embodiment of the present disclosure. This embodiment comprises an inlet tube in fluidic communication with an electro-mechanical device. The electro-mechanical device may be detachable from the inlet tube. The electro-mechanical device may comprise a pump, heat exchanger, temperature sensor, and other temperature-regulating equipment. This embodiment further comprises an outlet tube in fluidic communication with the electro-mechanical device. Each of the inlet tube and outlet tube are in fluidic communication with a plurality of microtubes, each microtube substantially parallel with one another and alternating between a microtube in fluidic communication with the inlet tube and a microtube in fluidic communication with the outlet tube. The microtubes in fluidic communication with the outlet tube may contain air at a lower pressure than the outside environment, thus sucking warm air into the microtubes and into the outlet tube. The microtubes in fluidic communication with the inlet tube may contain air at a higher pressure than the outside environment, thus releasing cool air to the wearer of the garment. In one embodiment, the inner diameter of each microtube may be approximately 1-2 mm. The length of each microtube may be approximately 1 m. The number of microtubes for air supply and suction may be approximately 100 (50 for supply and 50 for suction). The inner diameter of the inlet and outlet tubes may be approximately 5 mm. The length of the inlet and outlet tubes may be approximately 0.5 m. The flow rate at the inlet or outlet tube may be approximately 6.3-12.6×10⁻⁶ m³/s. The flow rate into each microtube may be approximately 12.6-25.2×10⁻⁶ m³/s. The pressure drop across the microtubes may be approximately 0.5-35.3 kPa. The pressure drop across the inlet and outlet tube may be approximately 0.2-6.4 kPa.

One embodiment of the present disclosure comprises a clothing system having two sub-systems—(1) thermoregulatory undergarments (TRUS) that distribute cooling or heating power around the body surface and (2) an electro-mechanical device (EMD). The EMD may be compact, flexible and attachable to clothing accessories (like a belt) for converting electrical energy to cooling or heating, sensing the wearer's skin temperature, controlling the cooling/heating function of the TRUS, and communicating with an HVAC control unit. The clothing system may be washable by detaching the EMD from the TRUS. In one embodiment, the additional weight is no more than 10% of standard business attire.

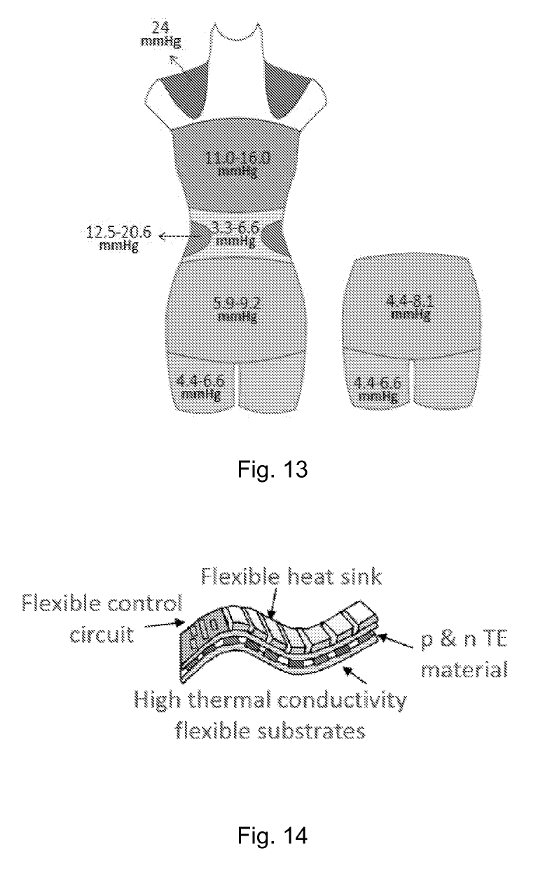

The TRUS have zones designed to stretch and conform to the wearer's body shape. Some portions of the TRUS containing microtubes may be less flexible than the stretch zones. In one embodiment, the TRUS may comprise of one or more stretch panels (without microtubes) that accommodate body motion. The stretch panels may be placed in the side of the torso, center back, or the crotch and hips. The pressure of the TRUS on the skin may be controlled for comfort. In one embodiment, the pressure of the TRUS may be configured as shown in FIG. 13. Heat supply to the wearer, or heat removal from the wearer, may be achieved by circulating warm or cold air through the microtubes embedded in the TRUS. The microtubes may be positioned next to the skin.

The EMD may comprise a flexible thermo-electric device, high efficiency induction charging element, and a fabric-IC interposer. The circulating air in the TRUS may be heated or cooled by pumping air through an energy conversion unit comprising flexible thermo-electrics. The EMD may further comprise a sensing and control unit configured to maintain an active communication with the HVAC system even when the battery has been drained.

The flexible thermo-electric device may be used to convert electrical energy to heating or cooling power. The EMD may comprise a flexible heat exchanger, for example, that can be incorporated into a clothing accessory, such as a belt. In one embodiment, the EMD may measure the skin temperature of the wearer and use the temperature to regulate the cooling/heating power of the thermo-electric device, the air circulation rate, and the HVAC control unit.

In another embodiment, the EMD may comprise an Energy Conversion unit (ECU), a Sensing & Control Unit (SCU) and a Power Collection and Storage Unit (PCSU) The ECU may convert electrical energy to cold/warm air flow for cooling or heating. The ECU may be composed of a thermoelectric (TE) unit, a heat exchanger and an air pump. The TE unit may cool down or heat up the heat exchanger through which air is cooled down or heated up and then pumped into the microtubes in the TRUS. The SCU may regulate the cooling/heating of the thermoelectric unit as well as the set-points of HVAC control unit based on the temperature sensors in TRUS. Since personal thermal management saves energy, the thermoregulatory function of the undergarments should first be fully utilized before activating and regulating the HVAC system. The PCSU may have a two-tier design for the SCU running on a constant power of 5-30 μW scavenged from an ambient far-field RF energy, and for the Energy Conversion Unit ECU running on an on-demand power of 5-40 W from induction and battery storage. FIG. 14 shows one design of an ECU having flexible thermoelectric devices and thermal ground planes. The flexibility of thermoelectric device can be achieved by using patchable high thermal conductivity inorganic substrates (for example, 5 mm×5 mm foot print of each device and then patch together multiple devices for the target energy delivery) or flexible polymer substrates. All passive elements such as antennas and matching networks in the PCSU may be implemented in the large-area fabrics to boost energy collection volume. Ferrites may be being integrated into fibers for better electromagnetic control and shielding.

Two-tier power discipline for the PCSU may be adopted with RF scavenging (5-40 μW) for SCU and induction charging for battery (5-40 W) in the ECU. As the SCU is on without need of the battery, ambient information from the body and HVAC is always available and adaptive algorithms may make best decision on the current scenario. The integrated circuits (IC) for the SCU may be less than 1 mm² to implement a microcontroller with ≅10K gate-equivalent (GE) and 1K embedded flash memory for digital circuits, as well as nonlinear diode/transistor analog circuits for energy conversion, voltage regulation and body temperature sensing. Induction charging may need a larger IC area (5-10 mm²) to handle up to 40 W of power, and the distributed design on belt or fabrics will be mostly dictated by thermal consideration to control the power distribution and regulate the charging to prolong battery life and to protect the battery from large mechanical strain. A lithium-sulfur rechargeable battery with energy capacity of 500 W·hour/kg may be approximately 80 g with an energy capacity of 40 W·hour, which is sufficient to sustain an hour of EMD operation without recharging. As the power of the SCU is scavenged and likely unstable, asynchronous design may help event-driven computing with high tolerance on voltage-induced timing variation. With further help of nonvolatile SRAM cells and flip-flops.

An interposer connecting IC and the functional fibers may be provided. The interposer may provide a fiber locking mechanism for reliability against wear and laundry cycles. To enable secure sewing, interposer may comprise a thinned silicon die. The die may contain a minimal number of contact pads (e.g., 2 or 4 pads) which may be laser drilled or deep reactive ion etched (DRIE) in the middle to sew with conductive yarns. The pitch of the buttonholes may be approximately 0.5 mm. Data and power distribution may be combined by load modulation. The interposer may contain multiple fabric layers for the electrical insulation and mechanical stability, as well as feel comfort.

In some embodiments, induction charging may be used to power the EMD. For example, to reach 40 W induction charging on garment from a chair with less than 5 W residual heat, a high-efficiency induction charging system must be used. For example, such an induction charging system may have a larger coil area (for example, 33 cm by 3 cm on a belt or 33 cm by 33 cm on the garment), higher frequency transmission (for example, higher than 13.56 MHz and with effective shielding but avoiding the water microwave absorption band), and including Fe₃O₄ ferrite composites in fibers to boost quality. A ferrite composite may perform better than patterned permalloy cores due to the higher anisotropy field and reduced Eddy current loss and has a ready path to integration with fibers. Furthermore, fabrics may have a bi-axial modulus, which can enable efficient induction coupling of two solenoids on ferrite-filled fabrics when the wearer sits in a chair. Combination of ferrite-embedded polymers and conductors may be used. With this combination, only a very narrow, specific band needs to be considered and highly effective fabric isolation structures can be designed against electromagnetic penetration to guarantee safety from the viewpoints of specific absorption ratio (SAR) and thermal comfort to meet the OSHA standards of less than 1 mW/cm² radiation to human body within the 30-900 MHz zone.

Although the present disclosure has been described with respect to one or more particular embodiments, it will be understood that other embodiments of the present disclosure may be made without departing from the spirit and scope of the present disclosure. Hence, the present disclosure is deemed limited only by the appended claims and the reasonable interpretation thereof

1. A wearable device for regulating a temperature of a wearer, comprising:
a fabric configured to be worn by the wearer; and
one or more microtubes woven in the fabric, each microtube having an inlet and an outlet, and each microtube configured to transport a gas through the microtube from the inlet to the outlet.

2. The device of claim 1, further comprising a pump having an intake and an outlet, the pump configured to move a gas, and wherein each microtube inlet is in fluidic communication with the outlet of the pump.

3. The device of claim 2, further comprising a microcontroller in electronic communication with the pump, wherein the pump is controllable by the microcontroller.

4. The device of claim 3, further comprising a temperature sensor in electronic communication with the microcontroller, and wherein the microcontroller is configured to regulate the pump according to a signal received from the temperature sensor.

5. (canceled)

6. The device of claim 1, wherein the temperature sensor is configured to be positioned near the skin of the wearer.

7. The device of claim 2, further comprising an energy conversion device, the energy conversion device in fluidic communication with each microtube outlet and the intake of the pump.

8. The device of claim 2, further comprising a battery configured to provide energy to the pump, microcontroller, and/or the temperature sensor.

9. The device of claim 3, wherein the microcontroller is configured to communicate with an environmental system.

10. The device of claim 2, wherein the intake of the pump is exposed to ambient air.

11. The device of claim 1, wherein each microtube outlet is exposed to ambient air.

12. The device of claim 1, wherein the one or more microtubes are formed from an elastic material.

13. The device of claim 1, wherein the fabric is conductive to heat.

14. The device of claim 1, wherein the microtubes are formed from a heat conductive polymer.

15. The device of claim 1, further comprising one or more resistive wires positioned in the fabric, wherein the one or more resistive wires are configured to heat the wearer.

16. (canceled)

17. (canceled)

18. (canceled)

19. The device of claim 1, wherein the microtubes are porous, and the outlet is a plurality of pores.

20. (canceled)

21. A fabric for temperature regulation comprising:
a plurality of warp yarns in a warp direction;
a plurality of weft yarns in a weft direction; and
a plurality of microtubes in a warp and/or weft direction of the fabric, each microtube having an inlet and an outlet, and each microtube configured to transport a gas through the microtube from the inlet to the outlet;
wherein the fabric is formed by interweaving the plurality of microtubes with the warp yarns and weft yarns as the fabric is being made.

22. The fabric of claim 21, wherein the plurality of microtubes are formed from an elastic material.

23. The fabric of claim 21, wherein one or more of the plurality of warp yarns or weft yarns are conductive to heat.

24. The fabric of claim 21, wherein the plurality of microtubes are formed from a heat conductive polymer.

25. (canceled)

26. The device of claim 21, wherein the microtubes are porous, and the outlet is a plurality of pores.
